(19) United States
(12) Patent Application Publication
(10) Pub. No.: US 2013/0197354 A1
(43) Pub. Date: Aug. 1, 2013

(54) MINIMALLY INVASIVE TREATMENT OF MITRAL REGURGITATION

(71) Applicants: Michael Maschke, Lonnerstadt (DE); Frederik Bender, Erlangen (DE); Thomas Redel, Poxdorf (DE)

(72) Inventors: Michael Maschke, Lonnerstadt (DE); Frederik Bender, Erlangen (DE); Thomas Redel, Poxdorf (DE)

(73) Assignee: Siemens Aktiengesellschaft, Munchen (DE)

(21) Appl. No.: 13/361,552

(22) Filed: Jan. 30, 2012

(51) Int. Cl. A61B 6/02; A61F 2/24; A61B 8/02; A61M 25/01; A61B 6/12; A61B 6/03

(52) U.S. Cl. 600/424

(57) ABSTRACT

A method of guiding a catheter during a treatment procedure includes acquiring scan data of a patient volume in position for the treatment procedure, registering volume data of the patient volume to the scan data, acquiring fluoroscopic data for the patient volume during the treatment procedure, and generating a volume representation of the fluoroscopic data during the treatment procedure. Generating the volume representation includes superimposing the fluoroscopic data on the volume data based on the registering. The volume representation of the fluoroscopic data is displayed, the volume representation showing a position of a mitral valve clip carried by the catheter relative to the patient volume.

### BACKGROUND

The present embodiments relate to imaging-based medical procedures.

Mitral valve insufficiency, or mitral regurgitation, is a heart condition in which the mitral valve fails to close during the ejection phase (systole). The mitral valve is positioned between the left atrium and the left ventricle of the heart. At the onset of the ejection phase (systole), the rising pressure in the ventricle normally causes the valve to close, thus sealing off the atrium. A mitral valve that fails to close completely causes a backward flow of blood from the left ventricle into the left atrium. An ultrasound examination is often conducted to diagnose the condition, with transesophageal echocardiography (TEE) used in some cases.

Mitral regurgitation has been addressed through a minimally invasive procedure in which the cusps, or segments, of the mitral valve are clamped together. In this procedure, a clip, such as the MITRACLIP from Evalve, Inc. (www.evalveinc.com), is positioned via a guide catheter while viewing with fluoroscopy and TEE imaging. The clamping procedure involves introducing a guide catheter into the right atrium using fluoroscopy imaging. The atrial septum is then pierced with the guidance of fluoroscopy and TEE imaging to introduce the clip into the left atrium. After firm clamping to the cusps of the mitral valve, the clip can improve closure of the mitral valve, thereby preventing the reverse flow of blood from the left ventricle to the left atrium.

The mitral valve procedure may be difficult due, in part, to limited visibility from the fluoroscopy and TEE imaging. The anatomy of the right atrium may not be sufficiently visible. A cardiologist may thus have difficulty assessing how far the guide catheter and the clip extend into the left atrium. Moreover, it is difficult to estimate where the optimal puncture site is located in the atrial septum.

If this puncture site is poorly selected, the clip may not be correctly positioned and mounted at the mitral valve segments.

### SUMMARY

By way of introduction, the embodiments described below include methods, systems, and computer readable storage media for guiding a catheter during a treatment procedure, such as a mitral valve treatment procedure. A volume representation is superimposed or overlaid on fluoroscopic data and displayed during the treatment procedure to facilitate guiding the catheter. The volume representation may be based on registration of volume data to scan data acquired in connection with the treatment procedure.

In a first aspect, a method of guiding a catheter during a treatment procedure includes acquiring scan data of a patient volume in position for the treatment procedure, registering volume data of the patient volume to the scan data, and acquiring fluoroscopic data for the patient volume during the treatment procedure. A volume representation on the fluoroscopic data is generated during the treatment procedure, in which the volume data is superimposed on the fluoroscopic data based on the registering. The method may further include displaying the volume representation on the fluoroscopic data, the volume representation showing a position of a mitral valve clip carried by the catheter relative to the patient volume.

In a second aspect, a system for guiding a catheter during a treatment procedure includes a memory in which volume data of a patient volume is stored, an x-ray imaging system operable to generate scan data of the patient volume and to generate fluoroscopic projection data for the patient volume, and a processor configured to transform the volume data via registration with the scan data and to generate a volume representation of the transformed volume data overlaid on the fluoroscopic projection data. The system may further include a display operable to display the volume representation during the treatment procedure, the volume representation showing a position of a mitral valve clip carried by the catheter relative to the patient volume.

In a third aspect, a computer readable storage medium has stored therein data representing instructions executable by a programmed processor for guiding a catheter in connection with a patient volume during a treatment procedure, the instructions including computer code to receive scan data of the patient volume, access a memory in which volume data of the patient volume is stored, register the volume data to the scan data, receive fluoroscopic data for the patient volume, superimpose the registered volume data on the fluoroscopic data, generate a volume representation of the superimposed volume data, and display the volume representation during the treatment procedure, the volume representation showing a position of a mitral valve clip carried by the catheter relative to the patient volume.

The present invention is defined by the following claims, and nothing in this section should be taken as a limitation on those claims. Further aspects and advantages of the invention are discussed below in conjunction with the preferred embodiments and may be later claimed independently or in combination.

### BRIEF DESCRIPTION OF THE DRAWINGS

The components and the figures are not necessarily to scale, emphasis instead being placed upon illustrating the principles of the invention. Moreover, in the figures, like reference numerals designate corresponding parts throughout the different views.

FIGS. 1A and 1B are flow chart diagrams of an example embodiment of a method for guiding a catheter during a mitral valve treatment procedure.

FIG. 2 is a block diagram of a system for implementing, and guiding a catheter during, a mitral valve treatment procedure according to one embodiment.

FIG. 3 is a flow chart diagram of a method, or a set of computer-executable instructions embodied on a computer readable medium, for generating a volume representation on fluoroscopic data, according to one embodiment.

### DETAILED DESCRIPTION OF THE PRESENTLY PREFERRED EMBODIMENTS

Systems, methods, and computer readable media are provided to support catheter-based treatment procedures, including, for example, procedures for minimally invasive treatment of mitral regurgitation. Three-dimensional (3D) or volume data is used to enhance a 2D display of fluoroscopic data captured during the treatment procedure. The guidance of one or more catheters during a treatment procedure is facilitated via the real-time display of images of atrial or other anatomy rendered from 3D data. The rendered images are generated during the treatment procedure based on a superposition of the volume data on the fluoroscopic data.

The volume data may be generated via computer tomography (CT) and utilized to assess the anatomy (e.g., atrial anatomy) before the treatment procedure. The volume data may thus be preoperative or other preliminary data acquired days or hours before the treatment procedure. The volume data is superimposed on fluoroscopic data captured by an x-ray system (e.g., a C-arm x-ray system) during the treatment procedure. The catheter, other instruments, and anatomy of, for instance, the left atrium may thus be clearly displayed during a mitral valve treatment procedure.

Although described in connection with mitral valve treatment procedures, the methods, systems, and computer readable media are not limited to the context of treatment procedures addressing mitral regurgitation. The methods, systems, and computer readable media are well suited for application in a variety of treatment procedures in which a catheter or other medical instrument is displayed relative to an anatomical volume during the procedure. The treatment procedures need not be considered minimally invasive, and may thus involve invasive elements or include invasive procedures, including, for instance, surgical procedures. The methods, systems, and computer readable media may be applied to improve the display of anatomical structures other than the left atrium of the heart. In mitral valve treatment procedure examples, such other anatomical structures include, for example, the mitral valve annulus.

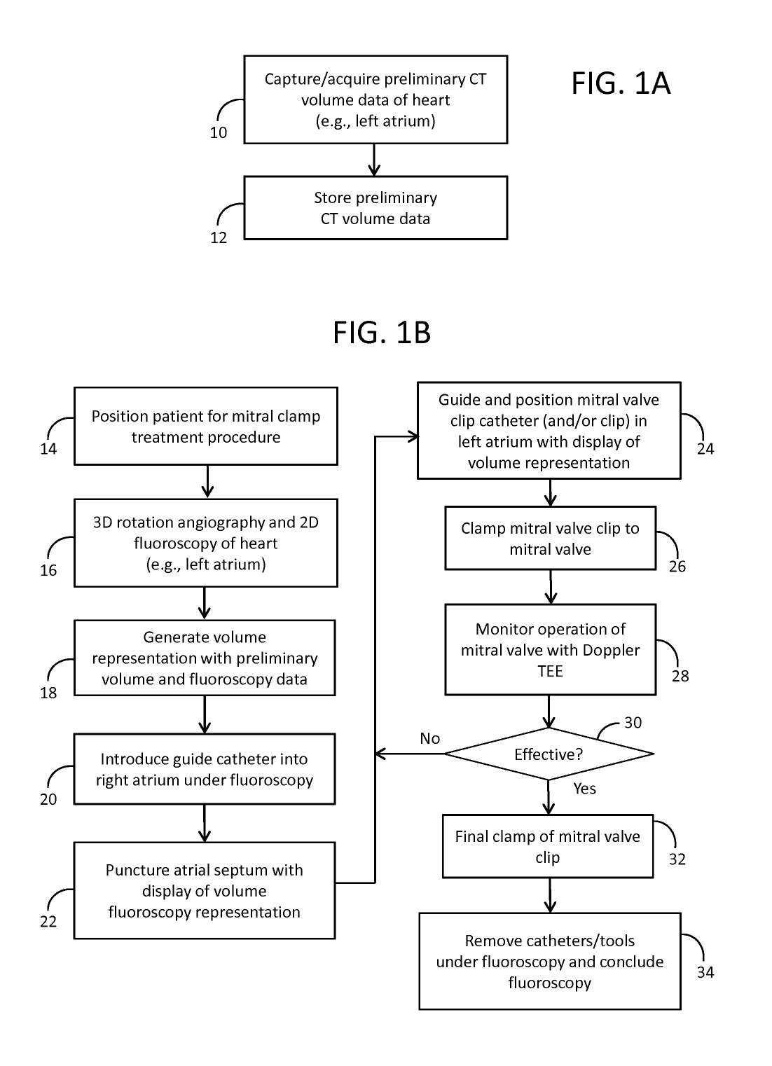

FIGS. 1A and 1B depict a method of facilitating the guidance of a catheter during a treatment procedure, such as a mitral valve treatment procedure. FIG. 1A depicts a number of imaging or scanning-related acts that may be implemented before the treatment procedure. For example, the acts in FIG. 1A may be implemented during a planning, diagnosis, or other preliminary session. The acts in FIG. 1A may alternatively be implemented as, and/or nonetheless considered to be, part of the treatment procedure. FIG. 1B depicts a number of imaging or scanning-related acts that may be implemented during, or as a part of, the treatment procedure. The acts of FIG. 1B may be implemented in real-time while the treatment procedure is executed.

In the example of FIG. 1A, preoperative or preliminary volume data of a patient volume is captured or acquired in act 10. The patient volume may include the heart or any portion thereof, such as the left atrium. In one embodiment, the preliminary volume data may be acquired via one or more computer tomography (CT) imaging or scanning techniques. Such techniques may be implemented using x-ray or other radiography equipment. Alternatively or additionally, the preliminary volume data is obtained via other imaging modalities, such as positron emission tomography (PET), single photon emission computed tomography (SPECT), and/or 3D magnetic resonance imaging (MRI). The preliminary volume data may be obtained via any one or more imaging modalities.

The imaging or scanning that leads to the preliminary volume data may be implemented before the catheter-based treatment procedure. The catheter and other medical equipment involved in the treatment procedure may not be present. The imaging or scanning may be implemented for purposes other than supporting or facilitating the treatment procedure. For example, the imaging or scanning may be implemented to assess the atrial anatomy. The assessment may be useful in diagnosing the extent or degree of mitral valve insufficiency or regurgitation. For example, the CT or MRI data is acquired prior to the treatment procedure, such as just prior to (same day) or during a previous appointment on a different day. The data represents tissue, and may be in a high resolution.

The preliminary volume data may alternatively be obtained solely for the purpose of supporting the treatment procedure. The imaging or scanning that generates the preliminary volume data may thus be implemented commensurate or otherwise in connection with the treatment procedure.

The preliminary volume data, such as CT volume data, is then stored in act 12 in a memory or other data store. The preliminary volume data may be stored in a database, a file, or any other data structure. The data storage need not be non-volatile, and may instead involve a volatile memory accessed during implementation of the treatment procedure and the disclosed methods.

The CT volume data may be computed using any image reconstruction or CT data processing technique. For example, the CT volume data may be generated from any number of x-ray projections or other 2D scans.

With reference to FIG. 1B, the catheter-based treatment procedure may begin in act 14 in which the patient is positioned for the procedure. The patient positioning includes disposing the patient volume (e.g., the heart) in position for further imaging or scanning. The patient volume may be positioned relative to x-ray and/or other imaging equipment so that a physician may view images of the catheter and the anatomy during the treatment procedure. In one example, the imaging equipment includes an x-ray system having a C-arm unit and a fluoroscopy display system, as described further below. The C-arm unit may be useful for orienting the x-ray equipment relative to the patient volume. Other embodiments may include fewer, additional or alternative imaging or display systems or units. For instance, the imaging equipment need not be integrated into a single system capable of generating fluoroscopic images.

Once the patient volume is in position for the treatment procedure, acquisition of scan data of the patient volume may begin in act 16. The scan data may be acquired using an imaging modality or system (or scanner) different from the imaging modality or system (or scanner) used to obtain the volume data in act 10. The imaging modality used in act 16 may be directed to, and configured for, supporting real-time imaging of the patient volume (e.g., under fluoroscopy). For example, the imaging modality used in act 16 may include an x-ray system configured to provide C-arm angiography and/or fluoroscopy. The imaging modality used in act 10 may include an x-ray system configured to provide 3D CT scan data. The imaging modality used in act 10 need not be directed to real-time image displays and, thus, may provide or support images of higher quality (e.g., higher resolution). The scan data acquired in act 16 may be referred to herein as “intra-treatment scan data” to distinguish the scan data from the preliminary volume data rather than to specify the exact timing of the acquisition. In some cases, the intra-treatment scan data may be acquired solely during the treatment procedure. Alternatively, the intra-treatment scan data may include data acquired before the treatment procedure is started.

The imaging or scanning of the patient volume to generate the volume data and/or the intra-treatment scan data may, but need not, include the introduction of a contrast agent. A low dose of the contrast agent may be used in some embodiments. The resulting low number of projections (e.g., a few projections) acquired in act 16 may suffice due to the information obtained via the preliminary scan in act 10, as described below. Notwithstanding the foregoing, either one or both of acts 10 and 16 may be implemented without the use of a contrast agent. The lack of a contrast agent may be useful for scan sequences or procedures involving a length of time beyond that typically supported by contrast agents.

The patient volume scanned in acts 10 and 16 may be selected or defined to include one or more anatomical markings or features. The preliminary volume data and the intra-treatment scan data may thus be representative of one or more common features. For example, the volume data and the intra-treatment scan data may include data representative of the spinal column or one or more vessel origins. The vessels may be selected based on the extent to which the origins are clearly visible. Alternatively or additionally, the imaging or scanning in acts 10 and 16 may also include the introduction of one or more objects into (or near) the patient volume, including, for instance, pins, metal balls, or other solid objects. Such objects (e.g., fiducials) may be used as synthetic markings during the imaging or scanning process to support transformations, alignment, and other image registration or processing, as described below. The volume data and the intra-treatment scan data may thus include respective representations of the object(s), such that registration of the volume data includes aligning the respective representations of the object(s).

The intra-treatment scan data generated in act 16 may include volume or 3D data, and/or a number of 2D slice data sets. In one embodiment, the C-arm unit may be used to implement 3D rotational angiography and generate such intra-treatment volume or 3D data. The intra-treatment volume or 3D data may be generated using one or more other imaging modalities and equipment. Alternatively or additionally, the C-arm unit may be used to generate intra-treatment data representative of multiple 2D slices. For example, data representative of at least two slices oriented along different planes may be acquired. The planes may be oriented less than 90 degrees apart. The slice data for the different planes may be acquired in succession or simultaneously. The C-arm unit may be configured with a biplane system to support such simultaneous acquisition. For example, the Artis zee™ biplane system commercially available from Siemens AG (Erlangen, Germany) may be used. Alternatively, projection data may be acquired.

The intra-treatment scan data may include 3D rotational data. Such data may be acquired in a native mode without contrast agent. Alternatively or additionally, such data may be generated via reconstruction of a mask acquisition of a subtracted angiographic protocol. Other techniques for obtaining native scans without contrast agent may be utilized.

The image acquisition in act 16 also includes the acquisition of fluoroscopic data for the patient volume. Fluoroscopic images of the patient volume may thus be displayed for the physician during the treatment procedure. The fluoroscopic data may be representative of the entire patient volume or any portion thereof. The fluoroscopic data may be obtained using the same imaging modality used to generate the intra-treatment scan data. For example, an x-ray system configured with a C-arm unit as described above may also include fluoroscopy equipment to generate the fluoroscopic data. Further details regarding the use and configuration of an x-ray system to support the acquisition of both the intra-treatment scan data and the fluoroscopic data, as well as the subsequent use of such data to support catheter-based treatment procedures, are set forth in commonly assigned U.S. patent application Ser. No. 12/881,925, the entire disclosure of which is hereby incorporated by reference.

A volume or 3D representation on the fluoroscopic data is rendered or otherwise generated in act 18 during the treatment procedure. The volume representation may be used to provide a display of the patient volume to the physician during the treatment procedure. The display may help the physician locate one or more anatomical structures, and guide one or more catheters or other instruments relative to such structures. Further details regarding an example involving the left atrium and the treatment of the mitral valve are provided below.

The preliminary volume data (stored in act 12) is accessed to support the generation of the volume representation on the fluoroscopic data. As described in further detail below, the preliminary volume data may be transformed or aligned with the intra-treatment scan data via one or more image registration procedures. The transformation may be useful because the volume data may be acquired before the treatment procedure (e.g., hours or days before the treatment procedure). The patient and, accordingly, the patient volume are thus not in the same position for the two imaging procedures. For example, the preliminary volume data may be acquired with the patient positioned in a manner well suited for one imaging modality, while the intra-treatment scan data may be acquired with the patient positioned to facilitate the treatment procedure. The coordinate systems of the two imaging modalities may differ in one or more other ways.

The image registration process may include identifying one or more similarities between the preliminary volume data and the intra-treatment scan data. Image processing may identify features. The similarity(ies) may be determined using a correlation, such as a minimum sum of absolute differences, cross correlation, autocorrelation, or other correlation. For example, a two or three-dimensional set of the volume data is translated and/or rotated into various positions relative to the intra-treatment scan data. The relative position with the minimum sum or highest correlation indicates a match, alignment, or registration location. The set of data may be a subset, such as a region of interest or a decimated set, or may be a full set. The set to be matched may be a subset or full set, such as correlating a decimated region of interest subset of data with a full set of preoperative data. Alternatively or additionally, a user may identify features. The relative positioning indicates a translation and/or rotation of one set of data relative to another set of data. The coordinates of the different volumes may be aligned or transformed such that spatial locations in each set representing a same tissue have a same or determinable location.

Once the preliminary volume data is transformed or registered to the coordinate system of the intra-treatment scan data, the preliminary volume data may be superimposed on the fluoroscopic data to generate the volume representation. The superposition on the fluoroscopic data may include an overlay process in which the preliminary volume data is overlaid on the fluoroscopic data. In some embodiments, the superposition is implemented based on data representative of one or more anatomical markings, such as the spinal column or vessel origins, in the fluoroscopic data. Thus, the superposition may be based on features or other characteristics extracted from the volume data, such as segmentations (e.g., the left atrium), or added markings, such as a planned puncture point. The added markings may be drawn by a user or inserted automatically through data analysis.

The fluoroscopic data may be registered or transformed to the common coordinate system. The registration may be based on using the same device as used for the intra-treatment scan data. Registration of the intra-treatment scan data may provide registration of the fluoroscopic data.

The volume representation on the fluoroscopic data may be displayed and used at one or more points during the treatment procedure. The volume representation need not be displayed or used throughout the treatment procedure. In the example of FIG. 1B, the treatment procedure may include introduction of a guide catheter in act 20 into the right atrium under fluoroscopy alone. The fluoroscopy display may be generated using a conventional 2D projection acquired via, for example, an x-ray system equipped with a C-arm unit. Alternatively, the volume representation on the fluoroscopic data may be displayed during the implementation of act 20 and/or otherwise throughout the treatment procedure.

The mitral valve treatment procedure may continue in act 22 in which a physician punctures the atrial septum using the guide catheter or an instrument guided thereby. The guide catheter may include an ultrasound transducer and/or other sensor to provide further guidance during these portions of the procedure. The ultrasound transducer may be coupled to an ultrasound system configured to provide transesophageal echocardiography (TEE). The information provided by the TEE images may be supplemented by a display of the volume representation on the fluoroscopic data during implementation of act 22. The volume representation may provide an indication of the depth of one or more instruments (e.g., the guide catheter) within the left atrium. The depth information may help avoid any undesired punctures or other damage (e.g., to the left atrial wall). The volume representation may also help the physician select a puncture site well suited for the remainder of the treatment procedure (e.g., the installation of a mitral valve clip).

Once the atrial septum is punctured, the atrial septum is pierced in this example to introduce the guide catheter into the left atrium. The conventional projection of the fluoroscopic data may be displayed and relied upon during this portion of the procedure.

The guide catheter may then be used to guide and position in act 24 another catheter, or clip delivery system, configured to install a clamp or clip on the mitral valve. The guidance and positioning of the mitral valve clip catheter in the left atrium may be facilitated by the display of the 3D information provided by the volume representation of the fluoroscopic data. TEE image data may also be displayed to facilitate the positioning of the mitral valve clip catheter.

The mitral valve clip catheter may include a mitral valve clip, such as the MitraClip® device commercially available from Abbott Laboratories (formerly Evalve, Inc.). The mitral valve clip may be deployed from the mitral valve clip catheter (or clip delivery system), and then positioned above or at the segments, or cusps, of the mitral valve. One or both of the volume representation of the fluoroscopic data and the TEE images may be used for guidance of the mitral valve clip.

The mitral valve clip is clamped to the mitral valve in act 26. Details regarding the construction and clamping of such clips are set forth in U.S. Patent Publications Nos. 2005/0149014 and 2006/0184203, the entire disclosures of which are hereby incorporated by reference. The construction and configuration of the mitral valve clip may vary in other mitral valve treatment procedure examples.

The treatment procedure may include the monitoring of the operation of the mitral valve clip in act 28. In one example, the operation is monitored via Doppler TEE images of the blood flow through the mitral valve. The efficacy of the mitral valve clip is determined in a decision block 30. If the mitral valve clip fails or is otherwise ineffective in reducing or eliminating the mitral regurgitation, then control returns to act 24 to re-position and re-install the mitral valve clip. If the mitral valve clip effectively reduces or eliminates the mitral regurgitation, then a final clamping of the mitral valve clip may be implemented in act 32.

Removal of the above-described catheters and any other tools or equipment used during the treatment procedure occurs in act 34. The removal process may be monitored under fluoroscopy. In some cases, the volume representation of the fluoroscopic data is displayed to facilitate this segment of the procedure. With all catheters and tools removed, the fluoroscopy and treatment procedure may be concluded.

The acts of the method shown in FIGS. 1A and 1B need not be implemented in the order shown. For example, the generation of the volume representation of the fluoroscopy data in act 18 may be implemented after the guide catheter is introduced into the right atrium. A number of the acts may be implemented concurrently or in parallel in whole or in part. For example, the generation of the volume representation on the fluoroscopy data may be implemented continuously with the acquisition of the intra-treatment scan data, and/or throughout the treatment procedure. Additional, fewer, or alternative acts may be included in the method.

The above-described methods may be applied to anatomy other than or in addition to the left atrium. For example, the patient volume may include the mitral valve annulus and any one or more other structures relevant to the treatment procedure. The preliminary volume data may include data markings or other elements representative of such structures so that fluoroscopic data of such structures may be superimposed.

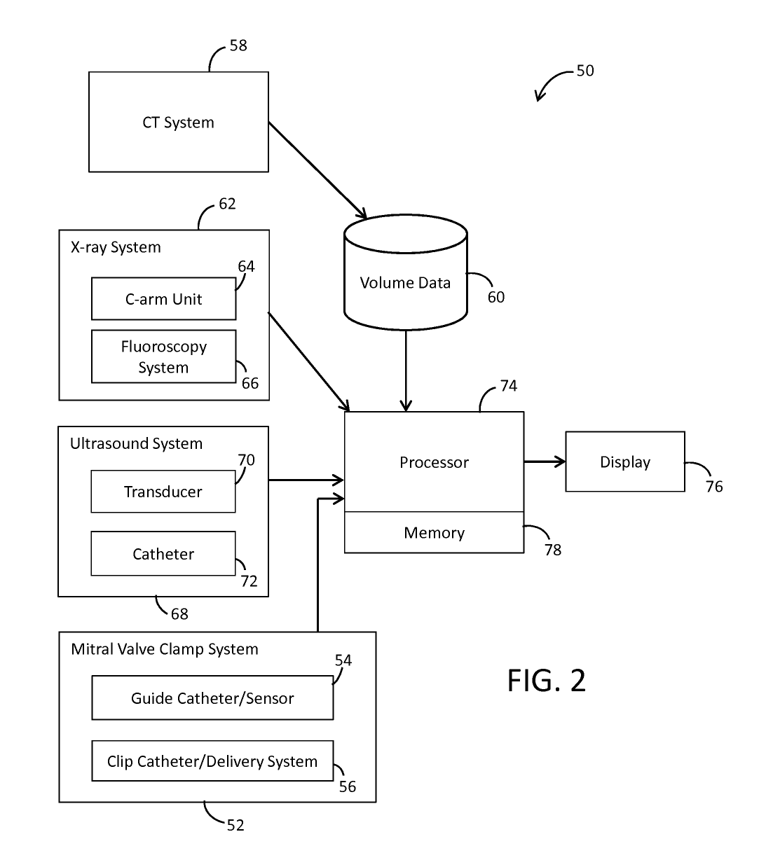

FIG. 2 depicts an exemplary system 50 configured to facilitate the implementation of a treatment procedure, such as a catheter-based treatment procedure. In this example, the system 50 includes a mitral valve clamp system 52 configured for use in a mitral valve treatment procedure. The mitral valve clamp system 52 includes a guide catheter 54 and a clip catheter or clip delivery system 56, the guidance and positioning of which may be facilitated by other components of the system 50. Other catheter-based treatment procedures are well suited for use with the system 50. The system 50 may be applied to a variety of treatment procedures. The system 50 need not include the catheter devices or other instruments guided during operation of the system 50. The system 50 may be adapted such that operation of one or more components of the system 50 implements one or more of the acts of the above-described methods.

The system 50 includes a CT imaging system 58 to generate preliminary volume data of a patient volume. The CT imaging system 58 may include, for example, an x-ray imaging system and one or more processors to construct the volume data from a number of x-ray scans. The CT imaging system 58 may instead be a different imaging modality. One or more of a variety of different scanners may be used. The CT imaging system 58 may include one or more imaging modalities or scanners. In some embodiments, the CT imaging system 58 includes a PET scanner, a SPECT scanner, and/or an MRI scanner.

The preliminary volume data is stored in a memory or other data store 60 for later use during the treatment procedure. In this example, the data store 60 includes a database. The configuration of the data store 60 may vary. The data store 60 may include any number of memories or data stores.

The CT system 50 and the data store 60 may be disposed in a different location(s) than one or more other components of the system 50. The CT system 50 need not be located at the site of the treatment procedure. For example, the CT system 50 may be located at a site at which diagnostic or planning information is captured for the patient. The CT system 50 may also be remotely located from the data store 60. The CT system 50 may be communicatively coupled or connected with the data store 60 and one or more other components of the system 50 via any communication technology.

The system 50 further includes an x-ray imaging system 62 operable to generate intra-treatment scan data of the patient volume and to generate fluoroscopic projection data for the patient volume. The x-ray imaging system 62 may include a C-arm unit 64 to generate the intra-treatment scan data, and a fluoroscopy system 66 to generate the fluoroscopic projection data. These and other components of the x-ray imaging system 62 may be integrated to any desired extent. The C-arm unit 64 may include a C-shaped structure having ends on which an x-ray source and a detector are mounted. The patient volume is disposed between the ends of the C-shaped structure. In one example, the C-arm unit 64 is the Zxiom Artis dTA DynaCT system commercially available from Siemens AG (Erlangen, Germany).

The C-arm unit 64 may be configured to implement three-dimensional rotation scans. For example, the C-shaped structure may be mounted to permit rotational movement about two axes for spherical motion. The C-arm unit 64 may thus be configured to generate 3D scan data of the patient volume. Alternatively or additionally, the C-arm unit 64 may be configured to implement multiple slice scans of the patient volume such that the scan data includes 2D scan data. The 2D or 3D scan data may be directed to any portion of the patient volume for which the preliminary volume data is acquired. The patient volumes scanned by the CT system 58 and the X-ray system 62 may overlap to any desired extent.

The fluoroscopy system 66 may be configured to provide fluoroscopic projection data in real-time during the treatment procedure. The fluoroscopy system 66 may include a fluorescent screen, an image intensifier, and an image sensor, such as a charge coupled device (CCD) (e.g., a CCD video camera), to generate the fluoroscopic projection data. One or more components of the image intensifier may be provided by the C-arm unit 64. The fluoroscopic projection data may be configured for display of real-time moving images. Such images may be used during one or more parts of the treatment procedure. During other parts of the treatment procedure, the preliminary volume data is superimposed on the fluoroscopic projection data to support a 3D view or rendering of the anatomy and the catheter devices and other instruments or treatment devices in the patient volume.

The system 50 may also include an ultrasound system 68 to provide TEE data or other echocardiographic information during the treatment procedure. The ultrasound system 68 may be configured for extracorporeal scanning. Alternatively or additionally, the ultrasound system 68 is configured for intra-cardial ultrasound. The ultrasound system 68 may include one or more transducers 70 carried on one or more catheter devices 72 to support intra-cardial ultrasound scanning. The ultrasound system 68 may include one or more processors and displays to process and present the echocardiographic information. The display(s) may be shared by one or more other components of the system 50.

The ultrasound system 68 is any now known or later developed ultrasound imaging system. Transmit and receive beamformers relatively delay and apodize signals for different elements of the transducer 70. B-mode, Doppler, or other detection is performed on the beamformed signals. A scan converter, memory, three-dimensional imaging processor, and/or other components may be provided.

The transducer 70 is a one-, two-, or multi-dimensional array of piezoelectric or capacitive membrane elements. In one embodiment, the transducer 70 is a handheld or machine held transducer for positioning against and outside of the patient. In another embodiment, the transducer 70 is part of a probe for use within the patient, such as a transesophageal probe. For example, the transducer 70 is a one-dimensional array of elements within or on the catheter 72 used for intervention or a different catheter. The transducer 70 is alternatively mounted on a guide or other catheter.

The ultrasound data is output in a polar coordinate or scan converted Cartesian coordinate format. Acoustic energy is used to scan a plane and/or volume. For example, a volume is scanned by sequentially scanning a plurality of adjacent planes. Any format or scan technique may be used. The scanned volume may intersect or include all of the patient volume. For example, the heart is scanned with ultrasound.

The catheter 72 is any now known or later developed catheter for intervention or other use within a patient. The catheter 72 is sized and shaped for use in the circulatory system, such as having a diameter of 10 French or less, but a length of a foot or more. The catheter 72 is adapted for insertion within the patient, such as through a vessel or vein for extending into a heart chamber. The catheter 72 may include guide wires or be inserted through another previously positioned housing or catheter. The catheter 72 may include an electrode, scalpel, balloon, stent, or other device for use during the treatment procedure.

The ultrasound system 68 may be integrated to any desired extent with the mitral valve clamp system 52. For example, the guide catheter 54 may include an ultrasound sensor that corresponds with the transducer 70 (or one of the transducers 70) of the ultrasound system 68.

The system 50 also includes a processor 74 in communication with one or more of the above-described components of the system 50 to process the data generated thereby and present the processed data via a display 76. For instance, the processor 74 may be configured to transform or otherwise process the preliminary volume data as described above. The processor 74 may access the data store 60 or otherwise receive the preliminary volume data provided by the CT system 58. The processor 74 may be configured to implement one or more image registration procedures to transform or align the preliminary volume data with the intra-treatment scan data provided by the x-ray system 62. Once the volume data is registered to the coordinate system of the intra-treatment scan data, the processor 74 may then generate a volume representation of the fluoroscopic projection data. The display 76 is then operable to display the volume representation during the treatment procedure as described above.

The intra-treatment scan data, the fluoroscopic projection data, and the ultrasound data may be obtained from the x-ray system 62 and the ultrasound system 68 via any communication technology. Alternatively or additionally, the processor 74 may be integrated with one or more of the x-ray system 62 and the ultrasound system 68 to implement one or more processing tasks of the x-ray system 62 and the ultrasound system 68 involved in the generation of such data.

The processor 74 is a general processor, central processing unit, control processor, graphics processor, digital signal processor, three-dimensional rendering processor, image processor, application specific integrated circuit, field programmable gate array, digital circuit, analog circuit, combinations thereof, or other now known or later developed device for determining position and/or generating images. The processor 74 is a single device or multiple devices operating in serial, parallel, or separately. The processor 74 may be a main processor of a computer, such as a laptop or desktop computer, or may be a processor for handling some tasks in a larger system, such as in an imaging system.

The system 50 may include a memory 78 in communication with the processor 74. The memory 78 may store data representative of the above-described preoperative and intra-treatment data, as well as fluoroscopic data, in one or more stages of processing. The memory 78 is a graphics processing memory, a video random access memory, a random access memory, system memory, random access memory, cache memory, hard drive, optical media, magnetic media, flash drive, buffer, database, combinations thereof, or other now known or later developed memory device for storing data or video information. The memory 78 is part of an imaging system, part of a computer associated with the processor 74, part of a database, part of another system, or a standalone device. The memory 78 may store one or more datasets representing the patient volume.

The memory 78 or other memory is a non-transitory computer readable storage medium storing data representing instructions executable by the programmed processor 74 for guiding a catheter during a treatment procedure. The instructions for implementing the processes, methods and/or techniques discussed herein are provided on computer-readable storage media or memories, such as a cache, buffer, RAM, removable media, hard drive or other computer readable storage media. Computer readable storage media include various types of volatile and nonvolatile storage media. The functions, acts or tasks illustrated in the figures or described herein are executed in response to one or more sets of instructions stored in or on computer readable storage media. The functions, acts or tasks are independent of the particular type of instructions set, storage media, processor or processing strategy and may be performed by software, hardware, integrated circuits, firmware, micro code and the like, operating alone, or in combination. Likewise, processing strategies may include multiprocessing, multitasking, parallel processing, and the like.

In one embodiment, the instructions are stored on a removable media device for reading by local or remote systems. In other embodiments, the instructions are stored in a remote location for transfer through a computer network or over telephone lines. In yet other embodiments, the instructions are stored within a given computer, CPU, GPU, or system.

Additional, fewer, or different components may be provided. For example, a network or network connection is provided, such as for networking with a medical imaging network or data archival system. One or more user inputs or other user interfaces may be provided.

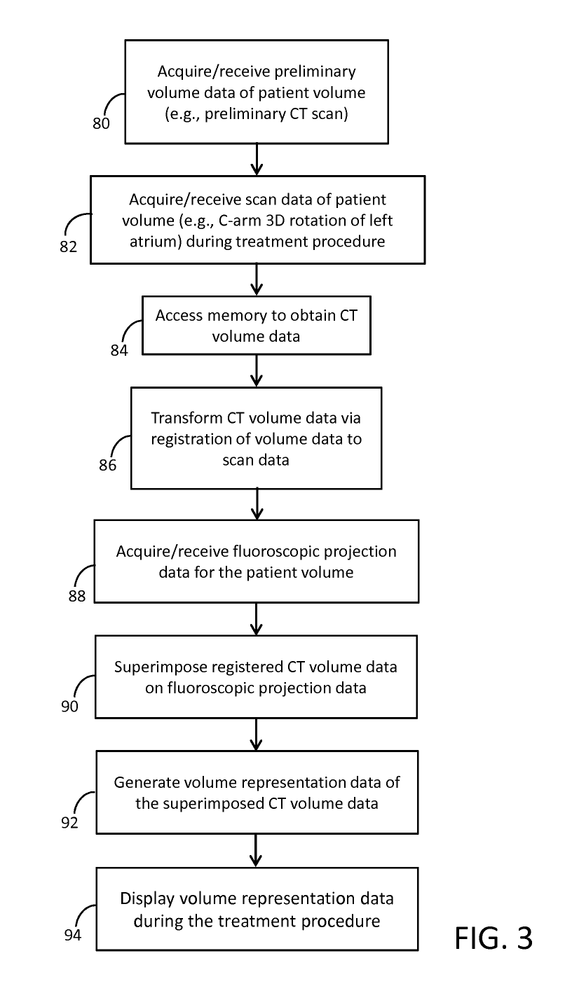

FIG. 3 depicts one example of acts implemented via instructions executable by the programmed processor 74 for guiding the above-described catheter-based treatment procedures. Data representative of the instructions may be stored in the memory 78 (FIG. 2). The acts may be implemented as a method involving one or more processors and one or more memories in addition or alterative to the processor 74 and the memory 78. One or more of the acts may be implemented as part of the methods described in connection with Figure

The instructions may include computer code to direct the processor 74 to perform the operations or acts shown in FIG. 3. Additional, fewer or different operations or acts may be implemented. For example, the preliminary volume data may already be stored and accessible to the processor 74.

The method begins in act 80 with the acquisition or reception of the preoperative or preliminary volume data generated by, for instance, a CT scan of the patient volume including, for instance, the left atrium. In one example, the preliminary volume data may be acquired directly from the imaging system that generates the volume data. The preliminary volume data is then stored in a database or other memory as described above for use during the treatment procedure. Alternatively, the preliminary volume data may be acquired from a database or other memory. The acquisition and/or storage of the preliminary volume data may occur during a diagnostic or planning session before the session in which the treatment procedure is performed.

In act 82, the intra-treatment scan data of the patient volume is acquired or received. The intra-treatment scan data may include the 3D C-arm rotational scan data and/or the C-arm slice data of the left atrium as described above. With the intra-treatment scan data available, the method may include accessing a memory in act 84 to obtain the preliminary volume data. The memory access may be implemented during the treatment procedure. The preliminary volume data is transformed or aligned with the intra-treatment scan data in act 86. The transformation may include one or more image registration processes to rotate or transform the volume data to the coordinate system of the scan data. Fluoroscopic data for the patient volume is acquired or received in act 88. The volume data (i.e., the volume data as aligned with the intra-treatment scan data via the registration) is superimposed on the fluoroscopic data in act 90. The superposition on the fluoroscopic data may include an overlay process, such as a 3D/2D overlay process in which a rendering of the volume data is superimposed on the projection of the fluoroscopic data. A volume representation on the fluoroscopic data is generated in act 92 and displayed in act 94.

The volume representation provides the real-time fluoroscopic data for determining a location of the catheter and/or an instrument (e.g., a mitral valve clip) carried by the catheter. The volume representation also provides the preliminary volume data for greater resolution and depth information for more detailed placement of the clip or other device. The intra-treatment scan data may be used for aligning the x-ray data to the preliminary volume data, allowing proper positioning of the fluoroscopic data relative to the preliminary volume data.

While the invention has been described above by reference to various embodiments, it should be understood that many changes and modifications can be made without departing from the scope of the invention. It is therefore intended that the foregoing detailed description be regarded as illustrative rather than limiting, and that it be understood that it is the following claims, including all equivalents, that are intended to define the spirit and scope of this invention.

1. A method of guiding a catheter during a treatment procedure, the method comprising:
acquiring scan data of a patient volume in position for the treatment procedure;
registering volume data of the patient volume to the scan data;
acquiring fluoroscopic data for the patient volume during the treatment procedure;
generating a volume representation on the fluoroscopic data during the treatment procedure, wherein generating the volume representation comprises superimposing the volume data on the fluoroscopic data based on the registering; and
displaying the volume representation on the fluoroscopic data, the volume representation showing a position of a mitral valve clip carried by the catheter relative to the patient volume.

2. The method of claim 1, wherein:
acquiring the scan data is implemented with a first imaging modality;
the volume data is obtained with a second imaging modality different from the first imaging modality.

3. The method of claim 2, wherein the first imaging modality is configured to provide C-arm angiography, and wherein the second imaging modality is configured to provide three-dimensional computed tomography.

4. The method of claim 1, wherein acquiring the scan data comprises obtaining C-arm slice data representative of multiple two-dimensional slices.

5. The method of claim 1, wherein acquiring the scan data comprises obtaining three-dimensional scan data via a C-arm rotational scan of the patient volume.

6. The method of claim 1, further comprising acquiring the volume data while the patient volume is not in the position for the treatment procedure.

7. The method of claim 6, further comprising administering a contrast agent to support acquisition of the scan data and the fluoroscopic data, and wherein acquiring the volume data is implemented without administration of the contrast agent.

8. The method of claim 1, wherein the treatment procedure comprises installation of the mitral valve clip via the catheter.

9. The method of claim 1, further comprising:
guiding the catheter into a left atrium of the patient volume with the displayed volume representation; and
clamping the mitral clip carried by the catheter onto a mitral valve of the patient volume.

10. The method of claim 1, further comprising introducing an object into the patient volume such that the volume data and the scan data include respective representations of the object, and wherein registering the volume data comprises aligning the respective representations of the object.

11. The method of claim 1, wherein the patient volume comprises a left atrium.

12. A system for guiding a catheter during a treatment procedure, the system comprising:
a memory in which volume data of a patient volume is stored;
an x-ray imaging system operable to generate scan data of the patient volume and to generate fluoroscopic projection data for the patient volume;
a processor configured to transform the volume data via registration with the scan data and to generate a volume representation of the transformed volume data overlaid on the fluoroscopic projection data; and
a display operable to display the volume representation during the treatment procedure, the volume representation showing a position of a mitral valve clip carried by the catheter relative to the patient volume.

13. The system of claim 12, wherein the x-ray imaging system comprises a C-arm unit.

14. The system of claim 12, wherein the x-ray imaging system is configured to implement three-dimensional rotation scans such that the scan data comprises three-dimensional scan data.

15. The system of claim 12, wherein the x-ray imaging system is configured to implement multiple C-arm slice scans of the patient volume such that the scan data comprises slice data representative of the multiple C-arm slice scans.

16. The system of claim 12, further comprising a computed tomography (CT) x-ray imaging system operable to generate the volume data.

17. The system of claim 12, further comprising an ultrasound system comprising a catheter and a transducer carried on the catheter and operable to generate transthoracic echocardiogram (TEE) data, and wherein the display is operable to display the TEE data.

18. A computer readable storage medium having stored therein data representing instructions executable by a programmed processor for guiding a catheter in connection with a patient volume during a treatment procedure, the instructions comprising computer code to:
receive scan data of the patient volume;
access a memory in which volume data of the patient volume is stored;
register the volume data to the scan data;
receive fluoroscopic data for the patient volume;
superimpose the registered volume data on the fluoroscopic data;
generate a volume representation of the superimposed volume data; and
display the volume representation during the treatment procedure, the volume representation showing a position of a mitral valve clip carried by the catheter relative to the patient volume.

19. The computer readable storage medium of claim 18, wherein the computer code to receive the scan data comprises further computer code to obtain C-arm slice data representative of multiple two-dimensional slices.

20. The computer readable storage medium of claim 18 wherein the computer code to receive the scan data comprises further computer code to obtain three-dimensional scan data via a C-arm rotational scan of the patient volume.

21. The computer readable storage medium of claim 18, wherein the patient volume comprises a left atrium.
